(19) United States
(12) Patent Application Publication
(10) Pub. No.: US 2006/0212124 A1
(43) Pub. Date: Sep. 21, 2006

(54) Knee joint prosthesis

(76) Inventor: Thomas Siebel, Saarbrucken (DE)

Correspondence Address: FRIEDRICH KUEFFNER, 317 MADISON AVENUE, SUITE 910, NEW YORK, NY, 10017, US

(21) Appl. No.: 10/554,807

(22) PCT Filed: Apr. 30, 2004
(86) PCT No.: PCT/EP04/04580

§ 371 Date: Oct. 28, 2005

(30) Foreign Application Priority Data
May 2, 2003 (DE) 102 20 034.7

(51) Int. Cl. A61F 2/38

(52) U.S. Cl. 623/20.31; 623/20.34

(57) ABSTRACT

The invention relates to a knee joint prosthesis comprising a femur part and a tibia part. The plane of the joint gap between said parts is inclined relative to the vertical axis extending from the center of the hip to the ankle according to the anatomical characteristics of the natural joint. According to the invention, at least the tibia part (9) is provided with a recess (19) for accommodating the anterior cruciate ligament. Said recess preferably encompasses a plate sector (20) that is formed within a plate (11) of the tibia part (9), which forms the tibia plateau.

The invention relates to a knee joint prosthesis comprising a femoral part and a tibial part, the plane of the joint gap between said parts being inclined relative to the vertical axis extending from the midpoint of the hip to the ankle in accordance with the anatomical characteristics of the natural joint.

An anatomical knee prosthesis of this kind is disclosed in DE 100 62 715 A1. It is a considerable improvement on previous knee joint prostheses in which the plane of the joint gap extends perpendicular to said axis and which are therefore only partially adapted to the actual anatomical circumstances.

Upon implantation of the latter prostheses also known from DE 100 62 715 A1, the anterior cruciate ligament is removed. Resulting impairment of the function of the artificial knee joint has to be taken into account.

The object of the present invention is to make available a novel knee joint prosthesis of the aforementioned type which, compared to the known prosthesis of this kind, can assume the function of the natural, healthy knee joint to a still greater extent.

The knee joint prosthesis according to the invention, by which this object is achieved, is characterized in that at least the tibial part has a recess creating space for the anterior cruciate ligament.

This inventive solution advantageously means that a knee joint prosthesis adapted to the inclination of the natural joint gap can be used in combination with a cruciate ligament prosthesis or with a preserved anterior cruciate ligament and, consequently, that the prosthesis can be made more like the natural knee joint in terms of its function. The possibility of preserving the anterior cruciate ligament or of introducing a corresponding cruciate ligament transplant means it is possible in particular to avoid instabilities during bending of the knee. In the healthy knee, gentle bending is associated with an internal rotation of the tibia about its longitudinal axis, with swiveling of the ankle bone, the medial femoral condyle migrating forward on the medially and dorsally downward sloping tibial plateau and migrating upward in accordance with the inclination of the plateau. The lateral condyle, by contrast, moves down the plateau, as a result of which the relative position of the femur and the tibia to one another changes in the direction of genu valgum. The anterior cruciate ligament ensures that this natural pattern of movement is retained even after implantation of the knee joint prosthesis and that there is no inverse rotation of the tibia, as happens in the absence of guidance by the anterior cruciate ligament, and therefore no genu varum position of femur and tibia.

In the preferred embodiment of the invention, the tibial part comprises a plate forming the tibial plateau, and the recess comprises a plate sector through which the cruciate ligament or a cruciate ligament implant can be guided or which frees an area for connection of the implant to the tibia.

The plate sector is preferably open toward the dorsal edge of the plate, so that, during implantation, a preserved cruciate ligament can pass laterally into the sector.

In one embodiment of the invention, the plate is connected to a hollow anchoring peg open toward the plate sector, and a through-opening for the cruciate ligament or cruciate ligament implant is formed in the wall of the anchoring peg. The hollow space of the anchoring peg can fill with spongy bone substance.

In a further embodiment of the invention, the plate sector is arranged between two anchoring pegs formed on the plate. The two anchoring pegs preferably adjoin the plate sector such that they are arranged as centrally as possible for stable anchoring in the shaft of the tibia. Surface lines of the anchoring pegs adjacent to the plate sector are preferably perpendicular to the edge of the sector, said surface lines extending through the tips of the pegs.

In a further embodiment of the invention, a further anchoring element can be formed on the plate opposite the dorsal edge opening of the plate sector, for example by a web projecting from the plate edge in the direction of the anchoring pegs. The latter anchoring element serves firstly for stabilizing the tibial part itself, by strengthening a part of the plate remaining between the ventral edge and the plate sector. Secondly, the web contributes to stabilizing the connection of the tibial part to the tibia.

The invention will now be explained in more detail on the basis of illustrative embodiments and with reference to the attached drawings which relate to these illustrative embodiments and in which:

FIG. 1 shows a first illustrative embodiment of a tibial part of a knee joint prosthesis according to the invention,

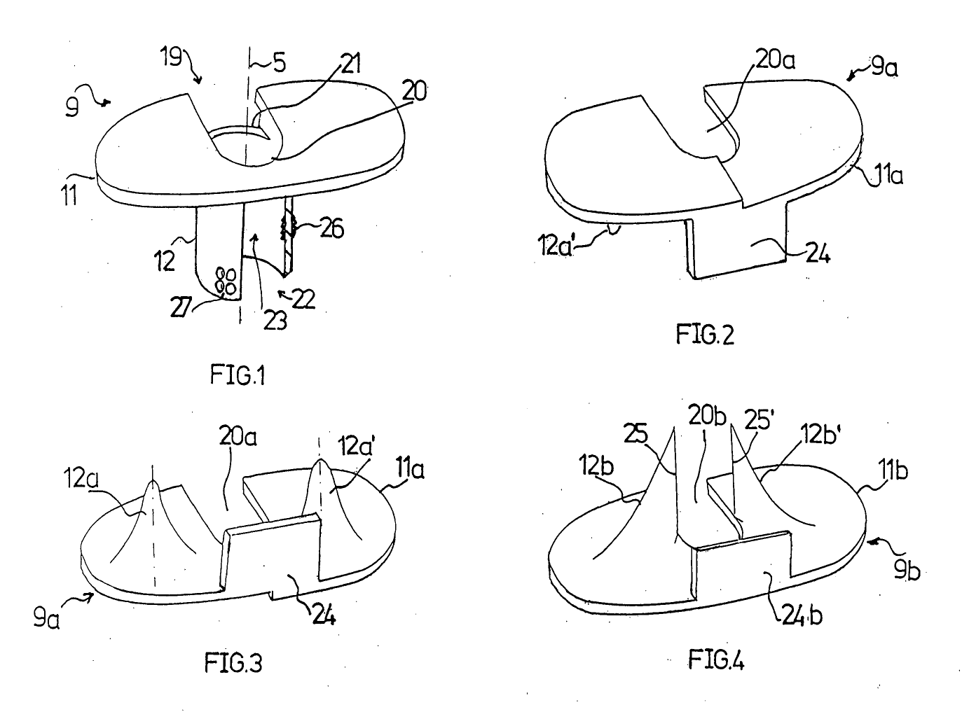

FIG. 2 shows a second illustrative embodiment of a tibial part of a knee joint prosthesis according to the invention, in a view obliquely from above,

FIG. 3 shows the tibial part from FIG. 2 in a view obliquely, from below,

FIG. 4 shows a further illustrative embodiment of a tibial part of a knee joint prosthesis according to the invention, in a view obliquely from below,

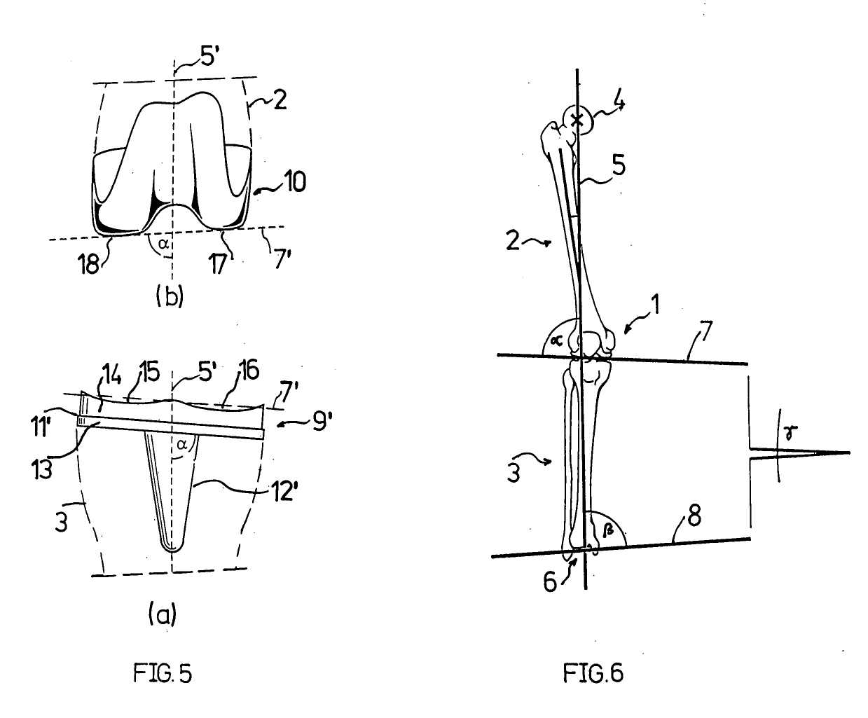

FIG. 5 shows a knee joint prosthesis according to the prior art, and

FIG. 6 shows a diagram of the skeletal structure of the leg in order to explain the function of the prostheses according to FIGS. 1 through 5.

Reference is made first to FIG. 6 which shows a front view of the femur 2 and tibia 3 connected at the knee joint 1.

A vertical axis 5 running through the midpoint of the hip 4 extends through the ankle at 6.

An axis 7 running parallel to the plane of the tibial plateau and through the gap of the knee joint 1 intersects the axis 5 at an angle a which can deviate by up to 10° from a right angle and is on average 85°. The tibial plateau ascends laterally.

The axis 5 also intersects an axis 8 parallel to the tibial plateau on the ankle at an angle β which deviates by 2° to 3° from the right angle, the tibial plateau on the ankle sloping down to the side.

The axes 7 and 8 intersect each other at an angle γ which is on average about 7° to 8°.

It should be noted here that the tibial plateau slopes down to the rear by 3° to 7°.

FIG. 5 shows: a knee joint prosthesis which is known from DE 100 62 715 A1 referred to here and which takes account of the anatomical circumstances discussed above.

The knee joint prosthesis has a tibial part 9′ according to FIG. 5a, and a femoral part 10 according to FIG. 5b.

Protruding from a plate 11′ forming the tibial plateau of the tibial part 9′ there is a central, rotationally symmetrical anchoring peg 12′ whose longitudinal axis intersects the plane of the plate or an axis 7′ at an angle α. The plate 11, formed with a metal support part 13 and with a plastic attachment 14 on the plateau side, has indents 15 and 16 corresponding to condylar parts 17 and 18 of the femoral part 10. It will be appreciated that the axis 7′ reproduces an average course of a corresponding line on the surface of the tibial plateau.

Coresponding to the inclined tibial plateau sloping downward in the medial direction, the polycentric radii of the condylar parts 17 and 18 are of different sizes, and the medial condylar part 17 is farther forward, in relation to the axis 5, than the lateral condylar part 18.

Upon implantation, the anchoring peg 12′ is driven into the end face of tibia, its longitudinal axis 5′ coinciding with the axis 5, and the axis 7′ coinciding with the axis 7. The cap-like femoral part 10 adapted for implantation on the femur has its axis 5′ oriented with the axis 5. In relation to its position of implantation, the femoral part 10 in FIG. 5b is arranged turned through 180°.

Reference is now made to FIG. 1 where a tibial part 9 of a knee joint prosthesis is shown with a plate 11 forming the tibial plateau. Extending centrally from the plate 11 there is a substantially cylindrical anchoring peg 12 whose cylinder axis is to be oriented with the axis 5 and is at said angle with respect to the plane of the plate 11.

No surface structuring of the tibial plateau corresponding to the indents 15 and 16 from FIG. 5a is shown in FIG. 1.

The tibial part 9 has a recess at 19, which recess comprises a plate sector 20 opening dorsally at the edge of the plate 11, and a wall sector 21 of a cylinder wall 23 of the hollow anchoring peg 12. Part of the recess 19 is also an opening 22 in the wall 23 of the anchoring peg 12. The opening 22 extends along the full length and about a quarter of the circumference of the anchoring peg 12.

The anchoring peg has a structured surface coating which is indicated at 26 and which promotes the growth of bone tissue onto it. Alternatively, or in addition, the wall 23 of the anchoring peg can be broken through with a lattice formation, as is shown at 27, in order to allow bone tissue to grow through.

A cutting edge is expediently formed at the free end of the anchoring peg 12.

With the knee joint prosthesis implanted, the recess 19 permits implantation of an anterior cruciate ligament graft which runs through the opening 22 in the anchoring part 12 obliquely with respect to the plate 11 and continues through the plate sector 20 and the wall sector 21 to the femur where, in the notch between the condylar parts 17 and 18, it is joined to the bone tissue.

The hollow space in the anchoring part 12 can fill with spongy bone substance.

The prosthesis to a large extent assumes the function of the healthy knee since, even during bending of the knee, it ensures the naturally occurring internal rotation of the tibia.

In the following illustrative embodiments, identical parts or parts with identical action are designated by the same reference number, with addition of the letter a or b.

The illustrative embodiment in FIGS. 2 and 3 differs from the illustrative embodiment in FIG. 1 in that two approximately conical anchoring pegs 12a and 12a′ are provided in an off-centred position, without wall opening, and between them there is a plate sector 20a opening dorsally to the edge of a plate 11a. Opposite the opening of the plate sector 20a, a further anchoring element in the form of a web 24 is provided at the plate edge. The plate 11a is stepped so that the central plane of the plate is at the angle α with respect to the cone axis.

In the case where the anterior cruciate ligament has been preserved, the tibial part 9a can be implanted by means of the cruciate ligament being inserted laterally into the plate sector 20a.

In the illustrative embodiment in FIG. 4, two anchoring pegs 12b and 12b′ are formed directly on a plate sector 20b and have surface lines 25 and 25′ perpendicular with respect to the sector edge. The anchoring pegs 12b and 12b′ lying closer to one another can be longer than the pegs 12a and 12a′ of the illustrative embodiment in FIGS. 2 and 3 as they are driven more centrally into the bone shaft of the tibia.

1. A knee joint prosthesis comprising a femoral part (1) and a tibial part (9), the plane of the joint gap between said parts (9, 10) being inclined relative to the vertical axis (5) extending from the midpoint of the hip to the ankle in accordance with the anatomical characteristics of the natural joint (1), wherein at least the tibial part (9) has a recess (19) creating space for the anterior cruciate ligament.

2. The knee joint prosthesis as claimed in claim 1, wherein the tibial part (9) comprises a plate (11) forming the tibial plateau, and the recess (19) comprises a plate sector (20).

3. The knee joint prosthesis as claimed in claim 2, wherein the plate sector (20) is oped toward the dorsal edge of the plate.

4. The knee joint prosthesis as claimed in that claim 1, wherein the plate (11) is connected to a hollow cylindrical anchoring peg (12) open toward the plate sector (20) and its free end, and a through-opening (22) for the cruciate ligament or cruciate ligament implant is formed in the cylinder wall (23) of the hollow anchoring peg (12).

5. The knee joint prosthesis as claimed in claim 4, wherein the cylinder wall (23) is broken through with a lattice formation.

6. The knee joint prosthesis as claimed in claim 2, wherein the plate sector (20a; 20b) is arranged between two anchoring pegs (12a; 12a′; 12b; 12b′) formed on the plate (11a; 11b).

7. The knee joint prosthesis as claimed in claim 6, wherein the anchoring pegs (12b; 12b′) are arranged adjacent to the plate sector (20b).

8. The knee joint prosthesis as claimed in claim 7, wherein surface lines (25, 25′) of the anchoring pegs (12b, 12b′) adjacent to the plate sector (20b) are perpendicular to the edge of the plate sector (20b).

9. The knee joint prosthesis as claimed in claim 6, wherein a further anchoring element (24; 24b) is formed on the plate (11a; 11b) opposite the dorsal edge opening of the plate sector (20a; 20b).

10. The knee joint prosthesis as claimed in claim 9, wherein the further anchoring element is formed by a web (24a, 24b) projecting from the plate edge in the direction of the anchoring (12a, 12a′; 12b, 12b′).
